(19) United States
(12) Patent Application Publication
(10) Pub. No.: US 2018/0014983 A1
(43) Pub. Date: Jan. 18, 2018

(54) ABSORBABLE PAD

(71) Applicant: MAS Innovation (Private) Limited, Colombo (LK)

(72) Inventors: Daluwathumullagamage Praveen Nisal Jayasuriya, Colombo (LK); Achala Sajeeva Satharasinghe, Colombo (LK)

(21) Appl. No.: 15/548,724

(22) PCT Filed: Dec. 29, 2015
(86) PCT No.: PCT/SG2015/050511

§ 371 Date: Aug. 3, 2017

(30) Foreign Application Priority Data
Feb. 18, 2015 (GB) 1502721.2

(51) Int. Cl. A61F 13/49; A61F 13/515; A61L 15/46; A61L 15/26; A61F 13/66; A61F 13/494; A61L 15/58; A61F 13/84; A61F 13/505; A61L 15/44

(52) U.S. Cl. CPC A61F 13/49006; A61F 13/505; A61F 13/515; A61L 15/46; A61L 15/44; A61F 13/665; A61F 13/49453; A61L 15/58; A61F 13/8405; A61L 15/26; A61F 2013/8408; A61L 2300/404; A61F 2013/8414

(57) ABSTRACT

Disclosed herein is a liquid absorbing pad for use in clothing, comprising: a liquid permeable layer; a functional layer over the liquid impermeable layer; and a liquid impermeable barrier material, wherein the functional layer is capable of acquiring and distributing liquid and/or absorbing liquid and the liquid impermeable barrier material is bonded to at least the liquid impermeable layer and the functional layer around the periphery of said layers by a bonding means, provided that the bonding means is not stitching. The liquid absorbable pad may form part of a garment.

### FIELD OF THE INVENTION

The present invention relates generally to absorbable pads and, more particularly, to absorbable pads that are suitable to be attached to/form part of garments that absorb bodily fluids. For example, undergarments associated with menstruation and incontinence.

### BACKGROUND

Clothing may provide several functions, such as shaping the wearer's body, supporting parts of the body during exercise, and (in the case of undergarments) offering protection to outer clothing. However, while many people suffer from involuntary excretion of bodily fluids (such as urine, menstrual fluid, vaginal discharges, sweat, and breast milk), there are few items of clothing that have been designed to absorb such excretions that are functional, easy to wear and aesthetically pleasing.

For example, a woman who is menstruating will generally use a tampon or a sanitary pad, in addition to wearing an undergarment, to keep her outer garments from being soiled by bodily secretions and discharges. While the tampon or pad often absorbs all of the liquid flow, unexpected leaks can still occur. To avoid such leaks, she can instead choose to wear an adult “brief”, which offers a larger area of protection and may be particularly useful for women experiencing heavy menstrual flows. Such briefs may also be useful for men or women who have urinary incontinence. However, the current selection of adult briefs is unattractive because they are bulky and this makes it difficult or impossible to conceal their use under outer clothing, which may cause the wearer embarrassment. Further disadvantages of wearing adult briefs (and sanitary pads) include prolonged exposure to wetness, which may result in discomfort, irritant dermatitis, and/or infections. Further, the pads/tampons may be occasionally positioned incorrectly (e.g., too far up or back) and adult briefs may be wrapped too loosely, both resulting in leakage. In addition, these solutions are generally disposable, meaning that the environmental and economic costs can be significant.

Conventional “period underwear” is designed to reduce the risk of leakage by providing a leak-proof barrier (e.g., a laminated fabric) outside of the undergarment or a thick absorbent pad in the crotch area. However, this type of product is of limited use because the leak-proof barrier is normally in contact with the skin, so can cause skin irritation and the thick absorbent pad is bulky (so it is difficult to conceal) and uncomfortable to wear. Additionally, the leak-proof barrier and thick absorbent pad are generally designed to keep hold of the excreted liquid, leading to the risk of unpleasant odours, chafing, discomfort and an increased chance of infection (e.g. microbial infection).

As will be appreciated, the bulkiness of conventional products means that they do not allow the wearer to easily wear low-coverage undergarments. For example, absorbent pads must be placed into large, maximum-coverage undergarments that are capable of sufficiently containing them, while diapers and absorbent undergarments are generally large so as to adequately address the incontinence of the wearer. Therefore, someone who prefers thong-style or low-rise bikini underwear is often forced to choose between wearing preferred underwear, which risks leakage onto their outer clothing, or wearing a cumbersome and unattractive garment that would ensure that all leaks are prevented.

For individuals having overactive sweat glands, garments currently available to address such a condition have wicking and quick-dry properties that provide some assistance in moving the sweat from the body to the outer surface of the garment and increasing the speed with which it evaporates from the garment into the air. While this is helpful when a person does not mind having the exterior surface of their clothing wet, for example, when exercising, it does not help individuals who sweat in casual or more formal clothing and do not desire the wetness to show. Indeed, if wicking and/or quick-dry materials are used in undergarments, such as undershirts or underwear, such materials do nothing to prevent the outer clothing of the wearer from becoming wet.

Thus, there remains a need for washable, low-profile, low-coverage, breathable, quick-drying, odour-eliminating protective garments constructed from natural and/or synthetic fabrics and capable of capturing bodily fluids excreted or secreted from the body so that the wearer feels dry and the wearer's other clothing is protected.

### SUMMARY OF INVENTION

In a first aspect of the invention, there is provided a liquid absorbing pad for use in clothing, comprising a liquid impermeable layer, a functional layer over the liquid impermeable layer and a liquid impermeable barrier material, wherein the functional layer is capable of acquiring and distributing liquid and/or absorbing liquid and the liquid impermeable barrier material is bonded to at least the liquid impermeable layer and the functional layer around the periphery of said layers by a bonding means, provided that the bonding means is not stitching.

In a second aspect of the invention, there is provided a garment, the garment comprising a main fabric body configured to be worn by a subject and a liquid absorbing pad attached to an interior surface of the fabric body and extending over at least an area subject to bodily excretions, the liquid absorbing pad comprising:
a liquid impermeable layer;
a functional layer over the liquid impermeable layer; and
a liquid impermeable barrier material, wherein
the functional layer is capable of acquiring and distributing liquid and/or absorbing liquid and the liquid impermeable barrier material is bonded to at least the liquid impermeable layer and the functional layer around the periphery of said layers by a bonding means, provided that the bonding means is not stitching.

In embodiments of the first and second aspect of the invention:

(a) the functional layer may further comprise an antimicrobial substance. For example, the antimicrobial substance may be one or more substances selected from the group consisting of a silver-containing substance, titanium dioxide, a quarternary silane, hydrogen peroxide, triclosan and zinc pyrithione;

(b) the functional layer further comprises a substance that combats odour, for example the substance that combats odour may be one or more substances selected from the group consisting of nanoparticles with acid-neutralising pockets, high surface area mineral compositions, high surface area ceramic compositions and high surface area clay compositions;

(c) the bonding means that binds the liquid impermeable barrier material to at least the liquid impermeable layer and functional layer around the periphery of said layers may be a an adhesive or ultrasonic bonding (e.g. the adhesive may be an adhesive tape, liquid glue, or hotmelt powder glue. When the adhesive is an adhesive tape, the tape may be a single-sided, double-sided adhesive tape and said tape may have a single layer or multiple layers where said multiple layers may have one or more functions, such as barrier layers, elastic layers etc. When the adhesive is a liquid glue, the glue may be a hot melt glue, a liquid resin or combinations thereof (e.g. the adhesive may be a hot melt glue/liquid resin bonding by nozzle extrusion or liquid resin bonding by screen printing/template printing)), optionally, the adhesive or ultrasonic bonding is heat stable up to at least 190° C.;

(d) the functional layer may further comprise a stain-resistant component;

(e) the liquid impermeable layer and the functional layer may be bonded together using a single bonding means, provided that the bonding means is not stitching, optionally wherein the bonding means may be an adhesive or ultrasonic bonding (e.g. the adhesive may be an adhesive tape, liquid glue, or hotmelt powder glue. When the adhesive is an adhesive tape, the tape may be a single-sided or, more particularly, double-sided adhesive tape and said tape may have a single layer or multiple layers where said multiple layers may have one or more functions, such as barrier layers, elastic layers etc), optionally wherein the adhesive or ultrasonic bonding is heat-stable up to at least 190° C.;

(f) the liquid impermeable layer may be a material that is heat stable up to at least 190° C., optionally wherein the impermeable layer is made from a thermoplastic polyurethane film;

(g) the liquid impermeable barrier material is bonded to a peripheral region on an exposed surface of the liquid impermeable layer and is also bonded to a peripheral region on an exposed surface of the functional layer to form a liquid impenetrable barrier cuff.

In a further embodiment of the first and second aspect of the invention the functional layer is an acquisition and distribution layer and the liquid absorbing pad further comprises a liquid absorbing layer over the liquid impermeable layer and underneath the acquisition and distribution layer, where the liquid impermeable barrier material is bonded to at least the liquid impermeable layer and the acquisition and distribution layer around the periphery of said layers by a bonding means, provided that the bonding means is not stitching. For example,

(a) the liquid impermeable layer, the liquid absorbent layer and the acquisition and distribution layer have the same 2-Dimensional footprint;
the 2-Dimensional footprints of the liquid impermeable layer and the acquisition and distribution layer are essentially the same size; and
the liquid absorbent layer has a 2-Dimensional footprint that is smaller than the 2-Dimensional footprints of the liquid impermeable layer and the acquisition and distribution layer, optionally wherein 2-Dimensional footprint of the liquid absorbent layer is from 2 mm to 20 mm smaller around its periphery than the 2-Dimensional footprints of the liquid impermeable layer and the acquisition and distribution layer;

(b) the liquid impermeable layer, the liquid absorbent layer and the acquisition and distribution layer are bonded together using a single bonding means, provided that the bonding means is not stitching, optionally wherein the bonding means may be an adhesive or ultrasonic bonding (e.g. the adhesive may be an adhesive tape, liquid glue, or hotmelt powder glue. When the adhesive is an adhesive tape, the tape may be a single-sided or, more particularly, double-sided adhesive tape and said tape may have a single layer or multiple layers where said multiple layers may have one or more functions, such as barrier layers, elastic layers etc), optionally wherein the adhesive or ultrasonic bonding is heat-stable (e.g. up to at least 190° C.), optionally wherein a single piece of adhesive tape is used to bond the liquid impermeable layer or the acquisition and distribution layer to the liquid absorbent layer and the liquid impermeable layer to the acquisition and distribution layer adjacent to the periphery of said layers;

(c) the liquid impermeable layer is a material that is heat stable up to at least 190° C., optionally wherein the impermeable layer is made from a thermoplastic polyurethane film;

(d) the acquisition and distribution layer is a wicking layer (e.g. 100% polyester French terry fabric) and/or the liquid absorbent layer is a 100% polyester double terry fabric;

(e) the acquisition and distribution layer and/or the liquid absorbent layer further comprises an antimicrobial substance, optionally wherein the antimicrobial substance is one or more substances selected from the group consisting of a silver-containing substance, titanium dioxide, a quaternary silane, hydrogen peroxide, triclosan and zinc pyrithione;

(f) the liquid absorbent layer and/or the acquisition and distribution layer further comprises a substance that combats odour, optionally wherein the substance that combats odour is one or more substances selected from the group consisting of nanoparticles with acid-neutralising pockets, high surface area mineral compositions and a photocatalyst.

In yet further embodiments of the first and second aspects of the invention, the bonding means that binds the liquid impermeable barrier material to at least the liquid impermeable layer and acquisition and distribution layer around the periphery of said layers may be an adhesive or ultrasonic bonding (e.g. the adhesive may be an adhesive tape, liquid glue, or hotmelt powder glue. When the adhesive is an adhesive tape, the tape may be a single-sided, double-sided adhesive tape and said tape may have a single layer or multiple layers where said multiple layers may have one or more functions, such as barrier layers, elastic layers etc. When the adhesive is a liquid glue, the glue may be a hot melt glue, a liquid resin or combinations thereof (e.g. the adhesive may be a hot melt glue/liquid resin bonding by nozzle extrusion or liquid resin bonding by screen printing/template printing)), optionally wherein the adhesive or ultrasonic bonding is heat stable up to at least 190° C.

In still further embodiments of the first aspect of the invention, the liquid absorbing pad may comprise part of a garment comprising a main fabric body.

In still further embodiments of the first and second aspect of the invention, the liquid absorbing pad may form an integral part of the garment (when a garment forms part of the first aspect), optionally wherein part of the main fabric body is folded over and bonded to the liquid absorbing pad to form a seam (e.g. the liquid absorbing pad is bonded to the main fabric body by an adhesive tape, optionally wherein the adhesive tape is a double sided adhesive tape that binds the liquid impermeable barrier material to at least the liquid impermeable layer and functional layer/acquisition and distribution layer around the periphery of said layers to form the liquid absorbing pad and also binds the liquid absorbing pad to the main fabric body).

In yet still further embodiments of the first and second aspect of the invention, the functional layer/acquisition and distribution layer may further comprise a stain-resistant component, optionally wherein the main fabric body at least partly covers the functional layer/acquisition and distribution layer and said main fabric body further comprises a stain-resistant component.

In yet still further embodiments of the first and second aspect of the invention, the liquid absorbing pad and/or the garment are washable and reusable.

It has also been surprisingly found that the garment and pad are reusable up to a minimum of 30 (e.g. a minimum of 50 or 100) wash and dry cycles. The material selection and unique construction ensures that the garment and pad are washable without compromising on moisture management, anti-microbial and anti-odour functionality. Furthermore the integrity of the pad is maintained for a minimum of 30 (e.g. a minimum of 50 or 100) wash and dry cycles. This ensures that the gusset will not leak during the lifetime of the garment. For example the fabrics layers were specifically mentioned herein were chosen so that they remain chemically, thermally and mechanically stable throughout the intended lifetime of the product. That is, while undergoing a minimum of 30 (e.g. a minimum of 50 or 100) wash and dry cycles.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings.

FIG. 1 depicts a cross-sectional view of a liquid absorbing pad according to the current invention, fitted to a fabric body.

FIG. 2 depicts the areas where moisture is placed onto a gusset undergoing the gusset integrity test described in the experimental section below.

### DETAILED DESCRIPTION

In various embodiments, the present invention includes a liquid absorbing pad that may be fitted to a garment. The pad is leak-proof and enables the efficient transport of liquid from the surface of the pad to an internal portion thereof. When the pad is affixed to, or forms part of, a garment the resulting garment may also exhibit the properties associated with the liquid absorbing pad. Further, as the liquid absorbing pad of the current invention is thinner than conventional pads, the resulting garment may also be more attractive and more comfortable to wear than garments containing conventional pads.

The liquid absorbing pad and/or the entire garment may be treated with an antimicrobial agent and/or may be stain-proof and/or treated with a antimicrobial agent and/or be treated with an anti-odour agent. Additionally, the liquid absorbing pad provides sufficient and effective liquid absorption. Therefore, when the garment fitted with the liquid absorbing pad is in the form of an undergarment, there may be no need to use a disposable tampon/pad in conjunction with the undergarment.

Liquid Absorbing Pads

The current invention particularly relates to a liquid absorbing pad for use in clothing, comprising a liquid impermeable layer, a functional layer over the liquid impermeable layer and a liquid impermeable barrier material, wherein the functional layer is capable of acquiring and distributing liquid and/or absorbing liquid and the liquid impermeable barrier material is bonded to at least the liquid impermeable layer and the functional layer around the periphery of said layers by a bonding means, provided that the bonding means is not stitching.

The bonding means that binds the liquid impermeable barrier material to at least the liquid impermeable layer and functional layer around the periphery of said layers may be an adhesive or ultrasonic bonding (e.g. the adhesive may be an adhesive tape, liquid glue, or hotmelt powder glue). When the adhesive is an adhesive tape, the tape may be a single-sided, double-sided adhesive tape and said tape may have a single layer or multiple layers where said multiple layers may have one or more functions, such as barrier layers, elastic layers etc. When the adhesive is a liquid glue, the glue may be a hot melt glue, a liquid resin or combinations thereof (e.g. the adhesive may be a hot melt glue/liquid resin bonding by nozzle extrusion or liquid resin bonding by screen printing/template printing)). In certain embodiments, the adhesive or ultrasonic bonding is heat stable up to at least 190° C., which enables the pad to be used in a garment that can be washed and dried multiple times.

Unless otherwise specified, used herein, the term “heat stable” is intended to stipulate that there is no change in the physical state of the component in question after being subjected to the stated temperature for a period of time consistent with a machine drying cycle.

In certain embodiments, the liquid impermeable layer and the functional layer are bonded together using a single bonding means, provided that the bonding means is not stitching. Again, the bonding means may be an adhesive tape or ultrasonic bonding (e.g. a double-sided adhesive tape or any other tape as discussed herein), optionally wherein the adhesive tape is heat-stable (e.g. up to 190° C. or up to 95° C.), where the stability to heat also enables the pad to be used in a garment that can be laundered multiple times.

In certain embodiments, the liquid absorbing pad and/or the garment of the current invention will withstand at least 30 (e.g. a minimum of 50 or 100) machine wash and tumble dry cycles without change in overall appearance, integrity of the components and liquid management parameters as discussed herein. For example, the garment and pad may be reusable up to a minimum of 30 (e.g. a minimum of 50 or 100) wash and dry cycles. The material selection and unique construction ensures that the garment and pad are washable without compromising on moisture management, anti-microbial and anti-odour functionality (when one or both of the latter two are also present in the garment and/or pad). Furthermore the integrity of the pad may be maintained for a minimum of 30 (e.g. a minimum of 50 or 100) wash and dry cycles. This ensures that the gusset will not leak during the lifetime of the garment. For example, the fabrics layers specifically mentioned herein were chosen such that they remain chemically, thermally and mechanically stable throughout the intended lifetime of the product while undergoing up to 30 (e.g. up to 50 or up to 100) wash and dry cycles.

The liquid impermeable layer may be any material that is heat stable up to at least 190° C. For example, a liquid impermeable material may include one or more layers of a thermoplastic or thermoset film, where the thermoplastic or thermoset film is selected from one or more of the group consisting of polyurethane, polyester, polyolefin and silicone. Particular examples of liquid impermeable materials include layers made from a thermoplastic polyurethane film.

When discussed in embodiments herein, the functional layer is capable of acquiring and distributing liquid and/or absorbing liquid. The functional layer may be made from fibres or yarns made with fibres, where said fibres and yarns are selected from one or more of the group consisting of polyamide, polyester, polyolefin, polyurethane, polyacrylonitrile, natural cellulose, regenerated cellulose, regenerated cellulose derivatives (i.e cellulose acetate and cellulose triacetates), natural protein and regenerated protein. The functional layer may be produced using technologies such as knitting (warp knitting such as raschel Tricot, weft knitting such as circular or flat), weaving, non-woven methods (blow spinning, staple nonwoven, spun laid, air-laid, needle punched, thermal bonded, hydro-entangled, chemical bonded and so forth), electro-spinning, force-spinning etc. Additionally the functional layers may also include one or more of the coatings, treatments encapsulation or entrapments, which would enhance its liquid and moisture management functionality, such as rate of absorbency/wicking, absorption capacity, rate of spreading and distribution, one way liquid transport etc.

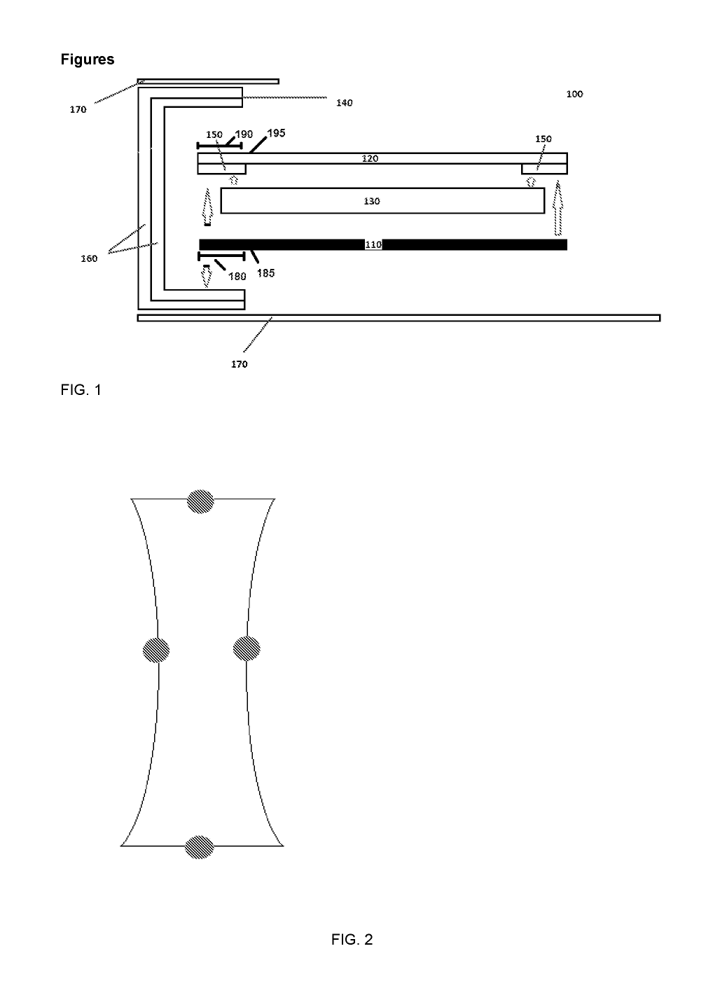

Further discussion of the materials is presented with respect to FIG. 1, which is an exploded diagrammatic representation of an absorbable pad according to the current invention, with only one portion of the impermeable barrier material (140) being shown to aid clarity. While the figure is discussed in respect of a liquid absorbable pad attached to a pair of underpants, this should not be considered to be limiting, as similar arrangements may be used with respect to other garments. In FIG. 1, a liquid absorbing pad 100 is attached to a fabric body 170, where the functional layer 120 is an acquisition and distribution layer and the liquid absorbing pad further comprises a liquid absorbing layer 130 over the liquid impermeable layer 110 and underneath the acquisition and distribution layer 120, where the liquid impermeable barrier material 140 is bonded to at least the liquid impermeable layer 110 and the acquisition and distribution layer 120 around the periphery of said layers by a bonding means 160. The bonding means 160 is not stitching and may be adhesive or ultrasonic bonding (e.g. the adhesive may be an adhesive tape, liquid glue, or hotmelt powder glue. When the adhesive is an adhesive tape, the tape may be a single-sided, double-sided adhesive tape and said tape may have a single layer or multiple layers where said multiple layers may have one or more functions, such as barrier layers, elastic layers etc. When the adhesive is a liquid glue, the glue may be a hot melt glue, a liquid resin or combinations thereof (e.g. the adhesive may be a hot melt glue/liquid resin bonding by nozzle extrusion or liquid resin bonding by screen printing/template printing)).

It will be appreciated that the impermeable barrier material 140 and bonding means entirely enclose the periphery of the functional layer 120 and the impermeable layer 110, thereby also providing a seal around the periphery of the liquid absorbing layer 130. It will be appreciated that in certain embodiments, the functional layer 120 may act as one or both of the acquisition and distribution layer and absorbable layer. When the functional layer acts as both the acquisition and distribution layer and absorbable layer, this may be accomplished by specific chemical treatment of the outer surface 195 of the functional layer 120, such that it enables liquid to be transported efficiently to the absorbable core material. Such chemical treatments will be known to the person skilled in the art and may be discussed in more detail hereinbelow

As depicted in FIG. 1 the liquid impermeable barrier material 140 may be bonded to a peripheral region 180 on an exposed surface 185 of the liquid impermeable layer 110 and may also be bonded to a peripheral region 190 on an exposed surface 195 of the functional layer 120 to form a liquid impenetrable barrier cuff. The benefits of this arrangement include reducing the leakage from the resulting pad onto clothing and also results in a durable pad that may be washed and reused. As depicted in FIG. 1, the particular arrangement described above may be achieved by the use of a C-fold arrangement of the impermeable barrier material 140. This C-fold arrangment entirely encloses the peripheral regions 180; 190 of the liquid impermeable layer 110 and functional layer 120.

It will be appreciated, that the following discussion of any materials herein is intended to be generally applicable, unless explicitly stated otherwise.

While not shown in FIG. 1, the liquid impermeable layer 110, the liquid absorbent layer 130 and the acquisition and distribution layer 120 have the same 2-Dimensional footprint. That is, the shape of each of the three layers is substantially identical when viewed from above/below the plane on which the layers lie. However, while the 2-Dimensional footprints of the liquid impermeable layer 110 and the acquisition and distribution layer 120 are essentially the same size, the liquid absorbent layer 130 has a 2-Dimensional footprint that is smaller than the 2-Dimensional footprints of the liquid impermeable layer 110 and the acquisition and distribution layer 120. For example, the 2-Dimensional footprint of the liquid absorbent layer 130 is from 2 mm to 20 mm (e.g. from 10 to 15 mm) smaller around its periphery than the 2-Dimensional footprints of the liquid impermeable layer 110 and the acquisition and distribution layer 120. This difference in size may allow all three layers to be unified into a single pad by means of a single bonding means, provided that this means is not stitching. For example, the bonding means may be a double-sided adhesive 150. In particular embodiments, the adhesive tape may be heat-stable (e.g. up to 190° C.). It is believed that using a single bonding means to unify the various layered components of the pad may help to reduce the likelihood of leakage. As noted hereinbefore, the double-sided tape may be replaced by any bonding means (e.g. as described hereinbefore), except by stitching.

It will be appreciated that any other possible arrangement of the layers that is technically feasible falls within the scope of this invention. For example, layers 110, 120 and 130 may have the same area/same 2D footprint, with the bonding being arranged as appropriate to the pad (e.g. absorbent layer 130 being sealed around its periphery with a single adhesive tape such that the liquid impermeable layer 110 and the acquisition and distribution layer 120 are attached to the absorbent layer via the adhesive, or two adhesive tapes are used—one to bond the acquisition and distribution layer 120 and the absorbent layer 130 together, while the other bonds the absorbent layer to the liquid impermeable layer 110). A further alternative example includes layers 110, 120 and 130, wherein the footprint of the layers increases in the order from 110 (smallest footprint), 130 and 120 (largest footprint). Again, any technically reasonable means to bond the layers together may be used.

The liquid impermeable layer 110 and the liquid impermeable barrier material 140 may comprise, consist of, or include any wholly or partially liquid-blocking material that is known. Preferably, the liquid impermeable layer is breathable, so that liquid may not pass through it, but gases (including water vapour) can do so. For example, the liquid impermeable layer and/or the liquid impermeable barrier material may be a liquid impermeable polymer (e.g. a thermoplastic polyurethane film). In certain embodiments, the liquid impermeable layer and/or the liquid impermeable barrier material may be a material that is heat stable up to at least 190° C.

In further embodiments, the liquid impermeable layer 110 may be a lightweight tightly knitted/woven fabric coated with SAP/hydrogel, or the liquid impermeable layer 110 may be a lightweight tightly knitted/woven fabric made using textile/SAP hybrid fibres. Alternatively, the liquid impermeable layer 110 may be a liquid-proof membrane (i.e. any liquid-proof membrane material supplied by Dingzing Advanced Materials Inc, Taiwan). When used in any pad or garment, such a liquid impermeable layer 110 may provide the advantage of being fully breathable in dry form, while providing an effective barrier material upon exposure to liquid. Furthermore, these materials may also enable the absorbable pad to dry more quickly than the use of a liquid impermeable polymer such as a thermoplastic polyurethane film.

The acquisition and distribution layer 120 faces to the wearer's body and serves to transport bodily fluids produced by the wearer's body to the liquid absorbing layer 130. In other words, the layer transports liquid from its surface that is in direct contact with the wearer to its internal surface that is in contact with the liquid absorbing layer 130. Suitable materials for the acquisition and distribution layer include polyamide, polyester, polyolefin, polyurethane, polyacrylonitrile, patural cellulose, regenerated cellulose, regenerated cellulose derivatives (i.e cellulose acetate and cellulose triacetates), natural protein and regenerated protein and any other wicking material known in the art. For example, the functional layer may be made from fibres or yarns made with fibres, where said fibres and yarns are selected from one or more of the group consisting of polyamide, polyester, polyolefin, polyurethane, polyacrylonitrile, natural cellulose, regenerated cellulose, regenerated cellulose derivatives (i.e cellulose acetate and cellulose triacetates), natural protein and regenerated protein. The functional layer may be produced using technologies such as knitting (warp knitting such as raschel Tricot, weft knitting such as circular or flat), weaving, non-woven methods (blow spinning, staple nonwoven, spun laid, air-laid, needle punched, thermal bonded, hydro-entangled, chemical bonded and so forth), electro-spinning, force-spinning etc. Additionally the functional layers may also include one or more of the coatings, treatments encapsulation or entrapments, which would enhance its liquid and moisture management functionality, such as rate of absorbency/wicking, absorption capacity, rate of spreading and distribution, one way liquid transport etc.

When the acquisition and distribution layer is a wicking material, the material may be naturally moisture-wicking and/or be treated to become moisture-wicking. For example, the wicking material may be 100% polyester fabric with French terry knit and a denier differential across the two faces of the fabric that assists in moving the liquid from the skin-facing side of the fabric to the internal surface that is in contact with the liquid absorbing layer. Other suitable wicking materials include blends of polyester, polypropylene and cotton.

An advantage associated with the use of a wicking layer having the triangular ridge structures of French Terry knitting facing the wearer's skin is that less surface area of the surface of the fabric comes into contact with the skin and therefore reduces any sensation of feeling wetness against the skin.

As an example, the acquisition and distribution layer may comprise a material that is 51% cotton and one or both of the inner and outer surfaces of acquisition and distribution layer 120 may be treated with a hydrophilic composition or material (e.g., polyethylene oxide, polyvinyl alcohol, polyacrylamide, poly acrylic acid, polyvinyl pyrrolidone, hydrophilic silicones, or hydrophilic polyurethanes) and/or a hydrophobic composition or material (e.g., silicones, polyfluoroalkylacrylates, polyacrylates, polyurethanes, or waxes) to create a net hydrophilic gradient over the acquisition and distribution layer. In other words, the surface in direct contact with the wearer's skin may be less hydrophilic (i.e., more hydrophobic) whereas the outer surface may be more hydrophilic. For example, the surface of the acquisition and distribution layer in direct contact with the wearer's skin may be treated with a hydrophobic material and/or the opposing surface may be treated with a hydrophilic material. This results in a combination of a “pushing” force generated by the hydrophobic properties of the surface in direct contact with the user's skin and a “pulling” force generated by the hydrophilic properties of the outer surface that may wick any moisture or liquid through the acquisition and distribution layer and away from the wearer. The hydrophilic and hydrophobic compositions may be applied to the acquisition and distribution layer using any conventional method.

Additionally, the differential capillary forces on either side can be created by the fabric structure where one side of the fabric has a smaller pore size in comparison to the opposite side of the fabric. Ideally, this pore combination creates funnel-like structures through the fabric in cross-section, where the liquid is pulled from the side with the larger pore size to the side with the smaller pore size, due to the differential capillary pressure.

The rate of wicking through the acquisition and distribution layer 120 may be controlled to be faster or slower. The rate may be set at a maximum rate of absorption of the acquisition and distribution layer to ensure that all, or a significant percentage of, the liquid is absorbed by the liquid-absorbing layer 130 and does not leak beyond the confines of the underpants. The rate of wicking may be controlled by the density, thickness, or composition of the acquisition and distribution layer 120 and/or by the amount and type of hydrophobic and/or hydrophilic material applied to said layer. In another embodiment, the rate of wicking may be set such that the surface of the acquisition and distribution layer in direct contact with the wearer feels “dry” or mostly dry to the wearer while the other surface may feel wet.

The liquid absorbing/absorbent layer 130 may comprise, consist of, or include any liquid absorbing material known in the art (e.g., cotton, a cotton blend, foam, a synthetic material, absorbent polymeric foam, a nanotechnology-based or -produced material, or any other moisture-absorbent material) and may have a weight of 50-500 g/m² (180-300 g/m²). For example, the liquid absorbing layer 130 may be made from an 80:20 blend of polyester:nylon fabric with a microfiber double terry knit. Other suitable materials include polypropylene or any cellulose-based fabric and their blends including cotton, bamboo etc.

In certain embodiments, the liquid absorbent layer 130 may be a 100% polyester double terry fabric. This material is approximately 90% air and so allows for a higher absorbent capacity, as moisture fills up the air gaps of the polyester terry fabric without significant expansion of the polyester fibres. This does not translate into significantly thicker pad.

In certain embodiments, the liquid absorbing layer 130 may be made from a blended fibre comprising two or more of SAP, hydrogel and polyester, or at least part (e.g. the surface facing towards the fabric body 170) of the liquid absorbing layer may have been treated with SAP and/or hydrogel. In these embodiments, which may be applicable to any absorbable pad in any type of garment, the use of these materials may result in increased liquid absorbing capacity, with a reduced thickness and weight for the pad, and in an improved dry feel on the surface of the acquisition and distribution layer 120 in contact with the wearer's skin, due to an increased affinity in the liquid absorbing layer 130.

As shown in FIG. 1, the liquid impermeable barrier material 140 may be bound to the periphery of the liquid impermeable layer 110 and the acquisition and distribution layer (i.e. functional layer) 120 by a bonding means. As shown in FIG. 1, the liquid impermeable barrier material 140 may be bound on one of its faces in a C-fold around the entire periphery of the liquid absorbing pad its other face may be bonded to the fabric body 170 of a garment.

In certain embodiments, said bonding means may be an adhesive or ulrasonic bonding (e.g. the adhesive may be an adhesive tape, liquid glue, or hotmelt powder glue. When the adhesive is an adhesive tape, the tape may be a single-sided, double-sided adhesive tape and said tape may have a single layer or multiple layers where said multiple layers may have one or more functions, such as barrier layers, elastic layers etc. When the adhesive is a liquid glue, the glue may be a hot melt glue, a liquid resin or combinations thereof (e.g. the adhesive may be a hot melt glue/liquid resin bonding by nozzle extrusion or liquid resin bonding by screen printing/template printing)), optionally the adhesive or ultrasonic bonding is heat stable up to at least 190° C. It will be appreciated that in other embodiments, the liquid-impermeable barrier material may be bound to all layers of the pad.

In certain embodiments, the functional layer (i.e. the acquisition and distribution layer) may further comprise an antimicrobial substance. For example, the antimicrobial substance may be one or more substances selected from the group consisting of a silver-containing substance, titanium dioxide, a quarternary silane, hydrogen peroxide, triclosan and zinc pyrithione.

In addition or alternatively, the functional layer (i.e. the acquisition and distribution layer) may further comprise a substance that combats odour. For example, the substance that combats odour may be one or more substances selected from the group consisting of nanoparticles with acid-neutralising pockets, high surface area mineral compositions, high surface area ceramic compositions and high surface area clay compositions.

In addition or alternatively, the functional layer (i.e. the acquisition and distribution layer) may further comprise a stain-resistant component.

Garment

A further aspect of the invention relates to a garment, the garment comprising a main fabric body configured to be worn by a subject and a liquid absorbing pad attached to an interior surface of the fabric body and extending over at least an area subject to bodily excretions, the liquid absorbing pad comprising a liquid impermeable layer, a functional layer over the liquid impermeable layer, and a liquid impermeable barrier material, wherein the functional layer is capable of acquiring and distributing liquid and/or absorbing liquid and the liquid impermeable barrier material is bonded to at least the liquid impermeable layer and the functional layer around the periphery of said layers by a bonding means, provided that the bonding means is not stitching.

In other words, a one or more of the liquid-absorbing pads as described hereinbefore may form part of a garment, whether integral or removable. Any garment that is intended to be in contact with a user's skin may be fitted with one or more liquid absorbing pads. For example, the garment may be outerwear, such as a shirt, a T-shirt, shorts, trousers/pants, leggings, running shorts, bicycle shorts, swimwear, yoga pants, body-shape-altering “stretch” pants, shorts etc. In particular, the garment may be sportswear or an undergarment (e.g. a bra or underpants, sport or “performance” underwear). One or more liquid absorbing pads may be fitted to cover a small area of the garment, which will generally be an area subject to the production of bodily excretions, such as the crotch area, the underarm area and the nipples of a pre- or post-partum female. Alternatively, the liquid absorbing pads may cover a major portion of the internal surface area of the garment, for example, a liquid absorbing in a pair of underpants may cover from 30-100% of the internal surface area of the garment. The level of internal surface area coverage of the liquid absorbing pad can be readily determined by the skilled practitioner based upon the intended use and the desired level of comfort of the wearer.

In a particular embodiment of the invention, the garment may be a pair of underpants. The underpants include a fabric body having a waist opening and a pair of leg openings defining a crotch area there between that covers some or all of the genital area of a wearer. The underpants contain an internal surface that is in contact with the wearer's skin (in this case the genital area of the wearer) and an external surface, where some or all of said external surface is not in direct contact with the wearer's skin. The underpants also contain a liquid absorbing pad (described in more detail below) that is attached to the interior surface of the fabric body. While it is possible to perform the attachment with stitching, it is preferred to attach the liquid absorbing pad using adhesive, adhesive tape or some other form of attachment.

More generally, as shown in FIG. 1, the liquid absorbing pad may be bonded to a garment using a C-fold arrangement of the fabric body 170 and the liquid impermeable barrier material 140 using an adhesive glue or, more particularly, a double-sided adhesive tape. In other words, part of the main fabric body is folded over and bonded to the liquid absorbing pad to form a seam. As shown in FIG. 1, the adhesive tape may be a double sided adhesive tape that binds the liquid impermeable barrier material to at least the liquid impermeable layer and functional layer/acquisition and distribution layer around the periphery of said layers to form the liquid absorbing pad and also binds the liquid absorbing pad to the main fabric body.

When the garment is a pair of underpants, the liquid absorbing pad may be disposed to cover the crotch area and the pad may extend over some or all of the crotch area and/or extend beyond the crotch area. It will be appreciated that any cut, size, style, colour, type of underpants are within the scope of the present invention.

The fabric body may contain one or more layers. For example, when the fabric body contains one layer the liquid absorbing pad can be attached to the crotch area of the fabric body. When the fabric body contains two layers, there is an inner fabric-body layer that wholly or partially contacts the body of the wearer and an outer fabric-body layer that is not in contact with the body of the wearer. In all embodiments, the outermost fabric-body layer may be one or more of various colours, patterns, or designs (e.g., black, white, pink, etc.) to provide choices to the wearer, though for a fabric body containing a single layer a dark colour may be preferred. When the fabric body comprises more than one layer, the innermost fabric-body layer may be a dark colour (e.g., black or dark grey) to help provide stain-resistance, so that any stain thereon is invisible or reduced in visibility or noticeability to the wearer. If the fabric body contains two or more layers, all, some, or none of the inner layer or layers may be visible to the wearer or other observer when the underpants are worn.

Any of the layers may be cut to the same size of the outer fabric-body layer or can be cut to less than the full size of said layer.

When the garment has a single fabric-body layer, the material may be made of cotton, a cotton blend, a synthetic material, an elasticized blend (e.g. SPANDEX) or any other material (e.g. natural or man-made textile). When the garment has two or more layers, the inner layers may be made of a thin fabric, while the outermost layer may be made of a thicker fabric, such as those described above. The inner fabric-body layer(s) may be cut to the full shape of the fabric-body or be cut to less than the full shape of the fabric-body. For example, an inner fabric-body layer may be cut to extend to cover only the crotch region. The inner fabric-body layer may also be densely stitched to the inner surface of the outer fabric-body layer to prevent leakage of fluids from the body of the wearer onto the outer surface of the underpants (or onto the wearer's outer clothes) and/or to any point on the inner surface of the underpants outside of the crotch area that may be felt or by the wearer. The underpants can be in any style. For example, the underpants may have a low-cut style where the waistband is lower than the wearer's waist (typically, at a location at or near the hips of the wearer) or the underpants can have a high cut, such that they include a section of fabric that extends above the waistband. It will be appreciated that any suitable height of waistband may be used.

As will be appreciated, the liquid absorbing pad and/or the garment may be washable and reusable, thereby helping to reduce environmental impact.

Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### EXPERIMENTAL SECTION

### One Way Transfer Coefficient Studies

Materials and Apparatus

The following materials and apparatus were used for the one way transfer coefficient studies:
MAS Generation 1 gusset composite sample (100 cm²)
THINX™ gusset composite sample (100 cm²)
Dear Kate™ gusset composite sample (100 cm²)
10 g samples of 0.9 saline solution
1.8 g swatches of standard tissue paper (100 cm²)
Weighing scale
Stopwatch
200 g cylindrical weight
Dropper (standard commercial dropper)

A circular area of 100 cm² was cut using a standard fabric sample cutting tool from each sample panty.

The MAS Generation 1 gusset composite material is constructed in line with FIG. 1. That is, layer 110 is a liquid impermeable, breathable polyurethane membrane, layer 120 is a 100% polyester double terry fabric and 130 is a 100% polyester French terry fabric. The bonding materials 150 and 170 are adhesive tape. The liquid impermeable barrier material 140 is a barrier tape capable of ensuring that the edges of the gusset are liquid impermeable. The composite was bonded together using 140, 150 and 170. No stitches were used in the process.

The THINX™ gusset is used in all commercially available styles (equivalent styles in terms of garment cut and fit being the “Hiphugger” or “Cheeky”). The gusset has the following composition:

INNER: 95% Cotton, 5% Elastane;

MIDDLE: Breathable PUL, 95% Cotton, 5% Elastane;

OUTER: 89% Nylon, 11% Elastane; and

TRIM: 100% Polyester.

Construction: the gusset is stitched onto the body fabric, no other form of bonding was used to unify the layers together or to the body fabric.

DearKate™—an equivalent style, such as the “Sport Hipster Full Lining” or “Ada Hipster Full Lining” was used in these tests. Composition: Body: 76% Nylon, 24% Lycra. Lining layers: 100% Micropolyester. Construction: gusset has been stitched onto the body fabric, no other form of bonding was used to unify the layers together or to the body fabric.

General Experiment Protocol

Dry weights for each layer of the gusset composite samples were determined. Each composite sample was then placed on a flat surface or an inclined surface (30 degrees to the horizontal). To the skin facing side (i.e. wicking/technical surface) of the gusset composite samples was added a 10 g sample of 0.9% saline solution (9 g of NaCl per litre of distilled water) in a single portion. This was done using a dropper positioned 10 mm above the skin facing side of the gusset composite samples.

A swatch of standard tissue paper (4.5″×4.5″ from Flora: www.floratissues.com) was then placed onto the skin facing side of the gusset composite samples. This was followed by placing a 200 g cylindrical weight on top of the standard tissue paper for compression. The gusset composite samples and the tissue papers were compressed for sixty seconds before the cylindrical weight was removed.

It will be appreciated that these experiments are interested in establishing the relative performance of the samples. As such, the tissue paper is immaterial, provided that the same tissue paper is used in each test.

The weight of the wet tissue paper and each layers of the gusset composite samples were then measured to determine their weight gains according to the following equation:

weight gain(g)=wet weight(g)−dry weight(g)

This was repeated five times for each gusset composite sample. The one way transfer coefficient of each sample was then calculated using the equation below:

One way transfer coefficient=% Weight gain on absorbent layer/% Weight gain on standard tissue layer (blot)

The percentage weight gains used above were calculated based upon the following equation:

% Wt gain=(Wet weight−Dry Weight)/Dry Weight×100

Flat surface test results

MAS Generation 1 gusset composite sample (100 cm²)

| % weight gain | % weight gain on standard | One way transfer |
|---|---|---|
| on absorbent | tissue (blot) | coefficient |
| 181.6 | 72.1 | 2.51 |
| 182.1 | 71.0 | 2.56 |
| 181.6 | 72.2 | 2.52 |

Average on way transfer coefficient: 2.53

THINX™ gusset composite sample (100 cm²)

| % weight gain | % weight gain on standard | One way transfer |
|---|---|---|
| on absorbent | tissue (blot) | coefficient |
| 196.9 | 66.3 | 2.96 |
| 196.1 | 66.5 | 2.94 |
| 197.0 | 66.7 | 2.95 |

Average on way transfer coefficient: 2.95

Dear Kate™ gusset composite sample (100 cm²)

| % weight gain | % weight gain on standard | One way transfer |
|---|---|---|
| on absorbent | tissue (blot) | coefficient |
| 218.4 | 163.0 | 1.34 |
| 219.2 | 164.1 | 1.34 |
| 218.3 | 163.5 | 1.34 |

Average on way transfer coefficient: 1.34

Inclined surface (30 degrees to the horizontal) test results

MAS Generation 1 gusset composite sample (100 cm²)

| % weight gain | % weight gain on standard | One way transfer |
|---|---|---|
| on absorbent | tissue (blot) | coefficient |
| 180.8 | 70.5 | 2.56 |
| 180.2 | 71.2 | 2.53 |
| 182.2 | 72.0 | 2.53 |

Average on way transfer coefficient: 2.54

THINX™ gusset composite sample (100 cm²)

| % weight gain | % weight gain on standard | One way transfer |
|---|---|---|
| on absorbent | tissue (blot) | coefficient |
| 197.0 | 66.5 | 2.96 |
| 196.5 | 66.5 | 2.95 |
| 196.9 | 66.3 | 2.96 |

Average on way transfer coefficient: 2.96

Dear Kate™ gusset composite sample (100 cm²)

| % weight gain | % weight gain on standard | One way transfer |
|---|---|---|
| on absorbent | tissue (blot) | coefficient |
| 218.2 | 162.5 | 1.34 |
| 219.5 | 164.5 | 1.33 |
| 218.2 | 162.8 | 1.34 |

Average on way transfer coefficient: 1.34

A higher transfer coefficient indicates that there is less moisture content on the layer touching the skin. Thus, higher numbers are generally favoured. However, the number is independent of certain modifications that that can be performed on the surface of the fabric, such as the triangular ridge structures using French Terry knitting. Modifications performed on the surface of the fabric can ensure that less surface area comes into contact with the skin and therefore reduces the sensation of feeling moisture. Thus, while the composite has a slightly lower transfer coefficient than that of the THINX™ composite, its French Terry ridge structure of the acquisition and distribution layer facing the skin's surface reduces contact with the skin and leads to a higher overall sense of dryness.

### Wicking Rate Studies

Materials and Apparatus

The following materials and apparatus were used for the one way transfer coefficient studies:
MAS Generation 1 gusset composite sample (100 cm²)
THINX™ gusset composite sample (100 cm²)
Dear Kate™ gusset composite sample (100 cm²)
5 g samples of 0.9 saline solution
1.8 g swatches of standard tissue paper (100 cm²)
Stopwatch
Dropper

If previously mentioned in the one-way transfer coefficient study, the materials are essentially identical to those previously described.

General Experiment Protocol

The gusset composite samples were placed on a flat or inclined surface (30 degrees to the horizontal). To the skin facing side (i.e. wicking/technical surface) of the gusset composite samples was added a 5 g sample of a 0.9% saline solution as a single portion. This was done using a dropper positioned 10 mm above the skin facing side of the gusset composite samples.

The saline solution was clear and all the fabrics were dyed black. The time taken for the saline solution droplets to completely disappear from the skin facing side was then recorded. This measurement was done by eye using two individuals and five attempts for each sample.

The rate of wicking was then calculated using the following equation:

Rate of wicking=20/saline solution droplet disappearance time (s)

Flat Surface Test Results

MAS Generation 1 gusset composite sample (100 cm²)

| Saline solution droplet disappearance time | |
|---|---|
| (s) | Rate of wicking |
| <1 | >20 |

THINX™ gusset composite sample (100 cm²)

| Saline solution droplet disappearance time | |
|---|---|
| (s) | Rate of wicking |
| >20 | <1 |

Dear Kate™ gusset composite sample (100 cm²)

| Saline solution droplet disappearance time | |
|---|---|
| (s) | Rate of wicking |
| <1 | >20 |

Inclined surface (30 degrees to the horizontal) test results

MAS Generation 1 gusset composite sample (100 cm²)

| Saline solution droplet disappearance time | |
|---|---|
| (s) | Rate of wicking |
| <1 | >20 |

THINX™ gusset composite sample (100 cm²)

| Saline solution droplet disappearance time | |
|---|---|
| (s) | Rate of wicking |
| >20 | <1 |

Dear Kate™ gusset composite sample (100 cm²)

| Saline solution droplet disappearance time | |
|---|---|
| (s) | Rate of wicking |
| <1 | >20 |

The higher the wicking rate, the faster a user will feel dry after an episode of leakage, particularly after a stress urinary incontinence episode. Therefore, as high a rate as possible is desired. When comparing the wicking rate and the one way transfer coefficient, the MAS composite had a higher transfer coefficient over DearKate™, but had a comparable wicking rate. The MAS composite had a higher wicking rate than THINX™ but a slightly lower one way transfer coefficient compared to THINX. However, on balance comparing both of these metrics, the MAS composite performs better because it does not compromise on wicking rate or transfer coefficient and also has the added benefit of having engineered triangular ridges on its surface, which reduces contact with the skin, so that the wearer feels dry. This is not a feature of THINX™ and DearKate™ do not possess such a ridge like structure.

### Saturated Capacity

Materials and Apparatus

The following materials and apparatus were used for the one way transfer coefficient studies:
MAS Generation 1 gusset composite sample (100 cm²)
THINX™ gusset composite sample (100 cm²)
Dear Kate™ gusset composite sample (100 cm²)
Beaker capable of holding 200 g of 0.9% saline solution
200 g samples of 0.9% saline solution
Stopwatch
Weighing scale

Unless otherwise stated, the materials are essentially identical to those described above.

General Experiment Protocol

Dry weights of the gusset sample composites were recorded. The samples were then completely submerged into 200 g of 0.9% saline solution for 150 seconds. Tweezers were used to hold the gusset composite samples in a submerged position. The gusset composite samples were then removed from the 0.9% saline solution and their wet weights were recorded immediately on an electronic weighing scale. This process was repeated five times for each of the gusset composite samples.

The % saturated gain for each of the samples was calculated using the following equation:

% saturated gain=(wet weight of gusset composite sample (g)−dry weight of gusset composite sample (g))/dry weight of gusset composite sample (g)

Test Results

| Gusset composite sample | % saturated gain |
|---|---|
| MAS Generation 1 gusset composite sample | 467.9 |
| (100 cm²) | |
| THINX ™ gusset composite sample (100 cm²) | 57.5 |
| Dear Kate ™ gusset composite sample | 50.2 |
| (100 cm²) | |

As illustrated above, the absorbent capacity of the MAS composite is higher. A polyester terry fabric which is approximately 90% air has been used as the absorbent layer. This lends itself to a higher capacity, such that moisture fills up the air gaps of the polyester terry fabric without significant expansion of the polyester fibres. This does not translate into a significantly thicker pad when it becomes wet.

### Gusset Integrity

Materials and Apparatus

The following materials and apparatus were used for the one way transfer coefficient studies:
MAS Generation 1 gusset composite sample in panty form with body fabric
THINX™ gusset composite sample in panty form with body fabric
Dear Kate™ gusset composite sample in panty form with body fabric
20 g samples of 0.9 saline solution

Materials are essentially identical to those discussed above.

General Experiment Protocol

Gusset samples were placed on a flat surface and 5 g samples of 0.9% saline solution were added to each extremity of the gusset (i.e. on the edges of the skin facing side/technical side, as illustrated in FIG. 2). The gusset edges were then observed from the body fabric side for gusset integrity failure. Any gusset integrity failure was verified using blotting paper. The experiment was repeated multiple times for each gusset sample. The experiment was performed five times on each sample.

Test Results

| Gusset sample | Observations |
|---|---|
| MAS Generation 1 gusset | Gusset did not leak liquid to external body |
| composite sample in | fabric. Gusset integrity maintained. |
| panty form (100 cm²) | |
| THINX ™ gusset composite | Gusset leaked liquid to the external body |
| sample in panty form | fabric. Gusset integrity not maintained. |
| (100 cm²) | |
| Dear Kate ™ gusset | Gusset leaked liquid to the external body |
| composite sample in panty | fabric. Gusset integrity not maintained. |
| form (100 cm²) | |

The THINX™ and DearKate™ gussets were designed to avoid leakage, while giving comfort to the wearer and both feature an overlock stitch, and other stitches, that attach the gusset to the body of the garment. While said gussets may provide these features, they do not effectively block all such leakages, as demonstrated above. In contrast, the embodiment of the current invention tested here prevents leakage from occurring. Indeed, the construction of the MAS gusset effectively prevents leakage through the direction perpendicular to the gusset as well as in the direction of the edges of the gusset. Therefore, the current gusset provides enhanced wearer comfort, while also providing enhanced protection from leakage.

1. A liquid absorbing pad for use in clothing, comprising:
a liquid impermeable layer;
a functional layer over the liquid impermeable layer; and
a liquid impermeable barrier material, wherein
the functional layer is capable of acquiring and distributing liquid and/or absorbing liquid and the liquid impermeable barrier material is bonded to at least the liquid impermeable layer and the functional layer around the periphery of said layers by a bonding means, provided that the bonding means is not stitching.

2. A garment, the garment comprising:
a main fabric body configured to be worn by a subject; and
a liquid absorbing pad attached to an interior surface of the fabric body and extending over at least an area subject to bodily excretions, the liquid absorbing pad comprising:
a liquid impermeable layer;
a functional layer over the liquid impermeable layer; and
a liquid impermeable barrier material, wherein
the functional layer is capable of acquiring and distributing liquid and/or absorbing liquid and the liquid impermeable barrier material is bonded to at least the liquid impermeable layer and the functional layer around the periphery of said layers by a bonding means, provided that the bonding means is not stitching.

3. The liquid absorbing pad according to claim 1, wherein the liquid impermeable barrier material is bonded to a peripheral region on an exposed surface of the liquid impermeable layer and is also bonded to a peripheral region on an exposed surface of the functional layer to form a liquid impenetrable barrier cuff.

4-7. (canceled)

8. The liquid absorbing pad according to claim, wherein bonding means that binds the liquid impermeable barrier material to at least the liquid impermeable layer and functional layer around the periphery of said layers is an adhesive or ultrasonic bonding (e.g. the adhesive is an adhesive tape, liquid glue, or hotmelt powder glue).

9-10. (canceled)

11. The liquid absorbing pad according to claim 1, wherein the liquid impermeable layer and the functional layer are bonded together using a single bonding means, provided that the bonding means is not stitching.

12. The liquid absorbing pad according to claim 11, wherein the bonding means is an adhesive or ultrasonic bonding, optionally wherein:
(a) the bonding means is a double-sided adhesive tape; and/or;
(b) the adhesive or ultrasonic bonding is heat-stable up to 190° C., such as up to 95° C.

13. (canceled)

14. The liquid absorbing pad according to claim 1, wherein the functional layer is an acquisition and distribution layer and the liquid absorbing pad further comprises a liquid absorbent layer over the liquid impermeable layer and underneath the acquisition and distribution layer, where the liquid impermeable barrier material is bonded to at least the liquid impermeable layer and the acquisition and distribution layer around the periphery of said layers by a bonding means, provided that the bonding means is not stitching.

15. The liquid absorbing pad according to claim 14, wherein:
the liquid impermeable layer, the liquid absorbent layer and the acquisition and distribution layer have the same 2-Dimensional footprint;
the 2-Dimensional footprints of the liquid impermeable layer and the acquisition and distribution layer are essentially the same size; and
the liquid absorbent layer has a 2-Dimensional footprint that is smaller than the 2-Dimensional footprints of the liquid impermeable layer and the acquisition and distribution layer.

16. (canceled)

17. The liquid absorbing pad according to claim 14, wherein the liquid impermeable layer, the liquid absorbent layer and the acquisition and distribution layer are bonded together using a single bonding means, provided that the bonding means is not stitching.

18-26. (canceled)

27. The liquid absorbing pad according to claim 1, wherein the liquid absorbing pad comprises part of a garment comprising a main fabric body.

28. The liquid absorbing pad according to claim, wherein the liquid absorbing pad forms an integral part of the garment.

29-31. (canceled)

32. The liquid absorbing pad according to claim 27, wherein the liquid absorbing pad forms a removable part of the garment.

33-35. (canceled)

36. The garment according to claim 2, wherein the liquid impermeable barrier material is bonded to a peripheral region on an exposed surface of the liquid impermeable layer and is also bonded to a peripheral region on an exposed surface of the functional layer to form a liquid impenetrable barrier cuff.

37. The garment according to claim 2, wherein bonding means that binds the liquid impermeable barrier material to at least the liquid impermeable layer and functional layer around the periphery of said layers is an adhesive or ultrasonic bonding (e.g. the adhesive is an adhesive tape, liquid glue, or hotmelt powder glue).

38. The garment according to claim 2, wherein the liquid impermeable layer and the functional layer are bonded together using a single bonding means, provided that the bonding means is not stitching.

39. The garment according to claim 2, wherein the functional layer is an acquisition and distribution layer and the liquid absorbing pad further comprises a liquid absorbent layer over the liquid impermeable layer and underneath the acquisition and distribution layer, where the liquid impermeable barrier material is bonded to at least the liquid impermeable layer and the acquisition and distribution layer around the periphery of said layers by a bonding means, provided that the bonding means is not stitching.

40. garment according to claim 39, wherein:
the liquid impermeable layer, the liquid absorbent layer and the acquisition and distribution layer have the same 2-Dimensional footprint;
the 2-Dimensional footprints of the liquid impermeable layer and the acquisition and distribution layer are essentially the same size; and
the liquid absorbent layer has a 2-Dimensional footprint that is smaller than the 2-Dimensional footprints of the liquid impermeable layer and the acquisition and distribution layer.

41. The garment according to claim 39, wherein the liquid impermeable layer, the liquid absorbent layer and the acquisition and distribution layer are bonded together using a single bonding means, provided that the bonding means is not stitching.

42. The garment according to claim 2, wherein the liquid absorbing pad forms an integral part of the garment.

43. The garment according to claim 2, wherein the liquid absorbing pad forms a removable part of the garment.
